(19) United States
(12) Patent Application Publication
(10) Pub. No.: US 2025/0257843 A1
(43) Pub. Date: Aug. 14, 2025

(54) PLATFORM FOR MOUNTING DEVICES

(71) Applicant: GAMBRO LUNDIA AB, Lund (SE)

(72) Inventors: Kishore Kumar AMPILI, Bangalore (IN); R. BIJU, Trivandrum (IN)

(21) Appl. No.: 18/858,010

(22) PCT Filed: Apr. 25, 2023
(86) PCT No.: PCT/EP2023/060790

§ 371 Date: Oct. 18, 2024

(30) Foreign Application Priority Data
Apr. 27, 2022 (IN) 202241024718

(51) Int. Cl. F16M 11/42

(52) U.S. Cl. CPC F16M 11/42

(57) ABSTRACT

The present invention relates to a platform for mounting devices. The platform comprises a frame made of one or more arms comprising through holes present near edges of the arms, and a plurality of support members configured to support the frame near the edges, each support member including a hollow shaft having internal threads passing via the through holes. The platform includes a plurality of spacers, each spacer being disposed on the through hole and comprising a bottom surface having a profile corresponding to a curvature of the frame, and a plurality of threaded plugs, each threaded plug passing through the spacer and the through hole. The threaded plug is secured with the internal threads of the hollow shaft positioned above the support member.

### FIELD OF INVENTION

The present invention generally relates to a platform for mounting devices, and more specifically relates to a platform for mounting devices in different configurations.

### BACKGROUND OF THE INVENTION

The subject matter discussed in the background section should not be assumed to be prior art merely as a result of its mention in the background section. Similarly, a problem mentioned in the background section are associated with the subject matter of the background section should not be assumed to have been previously recognized in the prior art. The subject matter in the background section merely represents different approaches, which in and of themselves may also correspond to implementations of the claimed technology.

There is an ever evolving need for solutions to apparatuses that utilize various electronic and/or motorized modules or auxiliary devices, especially those that are required to be rearranged, replaced, added to, or removed from a primary machine, such as for medical equipment. Some of the auxiliary devices may have a direct physical and electrical connection to the primary machine, while others may be indirectly attached and powered or controlled remotely by wires and cables.

In most medical settings, rearrangement of the auxiliary devices to a chassis and frame of the primary machine may need to occur prior to or during use of such primary machine. If during use of the primary machine, a need arises to change arrangement or configuration of the auxiliary devices or use of certain components, such an interruption may be inconvenient, may add extra time and cost to the overall setting, and may complicate delivery of care to a patient.

In medical domain, the auxiliary devices typically include a wide variety of components and accessories that are dependent on the patient's needs, user's preferences for operational setup, modular hardware, such as, Electrocardiogram (ECG) device, Pulse oximetry device, Temperature device, CO₂ device, console pumps, mast-mounted pumps, pump displays and controllers, diagnostic equipment, etc.

In a conventional setup, prior to carrying out a dialysis operation, it may be required to arrange a medical equipment into one of an extensive number of permutations that are meant to address the needs for the patient. Due to the extensive use of cables, blood conduits, pumps, pump displays, and other related devices that are commonly involved with such procedures, the cables and cords may become heavily intertwined and difficult to manage. This may lead to specific issues of operational inconvenience and time inefficiency for the operator.

As medical equipment often involve usage of a wide variety of the auxiliary devices that need to be controlled through the user interface of the primary machine, and given that any one of the auxiliary device may need to be switched out and replaced for various reasons, including but not limited a change in the delivery of therapy, or unexpected failure of the unit, there is a need to be able to quickly replace the auxiliary device so that the operator can attend to the delivery of medical support to the patient and minimize any downtime associated with the failure. When the auxiliary device is unable to be quickly switched out due to the routing of cables and wires, additional complications may occur.

Efforts have been made in the past to provide apparatuses for mounting and transport of the auxiliary devices with respect to the machine, in a medical setting. However, such apparatuses typically involve joining techniques such as welding or riveting of metal components, which pose a significant downside in quickly replacing or switching orientation of the auxiliary devices and lead to an increase in downtime associated thereto.

FIGS. 1A and 1B illustrate perspective views of a conventional platform 102 incorporating welded metal components. As shown in FIG. 1A, the platform 102 is joined to caster wheels 104 by welding. This restricts configuration of the auxiliary device mounted on the platform 102. For example, as illustrated in FIG. 1B when the platform 102 is present in an inverted position, i.e., the platform 102 is inverted by 180°, the caster wheels 104 are present above the platform 102. In such configuration, the platform 102 cannot be used for mounting devices.

Therefore, there is a need for a mounting platform that permit the user to simplify the reconfiguring, set up and management of the auxiliary devices. The present invention has been envisaged to addresses such deficiencies, and provides a platform for mounting devices in different configurations.

### OBJECTS OF THE INVENTION

An objective of the present invention is to provide a platform for mounting one or more devices in different configurations.

Another objective of the present invention is to provide a platform for mounting one or more auxiliary devices to be used with a primary machine/device, in different configurations.

Another objective of the present invention is to provide a platform for simplifying reconfiguring, set up and management of the auxiliary device used in conjunction with the primary machine, in a medical setting.

Another objective of the present invention is to provide a platform for mounting the auxiliary device used in conjunction with the primary machine, while reducing operational inconvenience and time inefficiency thereof.

Still another objective of the invention is to develop a platform for mounting the device in different configurations, potentially capable of being retrofitted with conventional medical apparatuses.

### SUMMARY OF THE INVENTION

An aspect of the present invention relates to a platform for mounting devices in different configurations. The platform includes a frame made of one or more arms having a plurality of through holes present near edges of the one or more arms, and a plurality of support members configured to support the frame near the edges of the one or more arms, each support member including a hollow shaft having internal threads passing through at least one of the plurality of through holes. The platform includes a plurality of spacers, each spacer being disposed on the at least one through hole and including a bottom surface having a profile corresponding to a curvature of the frame. The platform also includes a plurality of threaded plugs, each threaded plug passing through at least one of the plurality of spacers and the at least one through hole. The threaded plug is secured with the internal threads of the hollow shaft positioned above the support member, to enable coupling of the support member to the frame.

According to an embodiment of the present invention, the bottom surface of the at least one spacer has an arcuate profile corresponding to a rounded curvature of the frame.

According to an embodiment of the present invention, the platform further includes one or more base plates connected with opposite arms of the frame, for mounting the devices.

According to an embodiment of the present invention, the support member is a rolling member for supporting movement of the platform mounting the devices.

According to an embodiment of the present invention, the support member is a bearing pad for providing stability to the platform mounting the devices.

According to an embodiment of the present invention, the devices mounted on the platform include a primary device and at least one auxiliary device to be used in conjunction with the primary device.

According to an embodiment of the present invention, the frame is invertible, and the plurality of support members, the plurality of spacers, and the plurality of threaded plugs are connectable from a top side or a bottom side of the frame.

According to an embodiment of the present invention, a top surface of the threaded plug is flush with a top surface of the spacer.

### BRIEF DESCRIPTION OF THE DRAWINGS

The accompanying drawings constitute a part of the description and are used to provide a further understanding of the present invention. The drawings illustrate exemplary embodiments of the present disclosure and, together with the description, serve to explain the principles of the present disclosure.

FIGS. 1A and 1B illustrate perspective views of a conventional platform for mounting a device, incorporating welded metal components, in accordance with prior art.

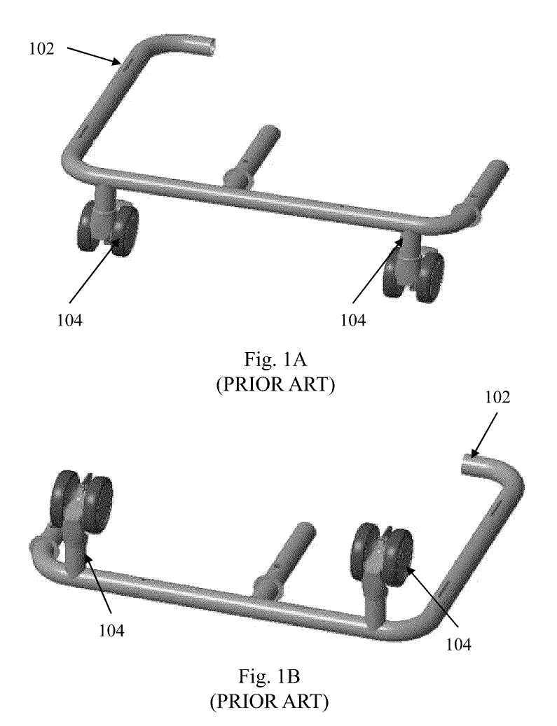

FIGS. 2A and 2B illustrate perspective views of a platform for mounting devices, in accordance with an embodiment of the present invention.

FIG. 3 illustrates perspective view of a threaded plug secured with a support member through a spacer of the platform, in accordance with an embodiment of the present invention.

FIG. 4A illustrates the spacer of the platform, in accordance with an embodiment of the present invention.

FIG. 4B illustrates a shaft of the support member of the platform, in accordance with an embodiment of the present invention.

FIGS. 5A and 5B illustrate different configurations of the platform mounting an auxiliary device to be used in conjunction with a primary device, in accordance with embodiments of the present invention.

### DETAILED DESCRIPTION OF THE INVENTION

The detailed description set forth below in connection with the appended drawings is intended as a description of various embodiments of the present invention and is not intended to represent the only embodiments in which the present invention may be practiced. Each embodiment described in this disclosure is provided merely as an example or illustration of the present invention. and should not necessarily be construed as preferred or advantageous over other embodiments. The detailed description includes specific details for the purpose of providing a thorough understanding of the present invention. However, it will be apparent to those skilled in the art that the present invention may be practiced without these specific details.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

The present invention relates to a platform for mounting one or more devices in different configurations. The platform includes components that are incorporated in a releasable manner. The components of the platform are reconfigurable, in that the components may be released and reconfigured to enable the platform to mount the devices in different configurations. The platform is capable of mounting the devices which serve as an auxiliary device to be used in conjunction with a primary device. For example, the platform may be used to mount an auxiliary device which is used in conjunction with a primary medical device, in a medical setting.

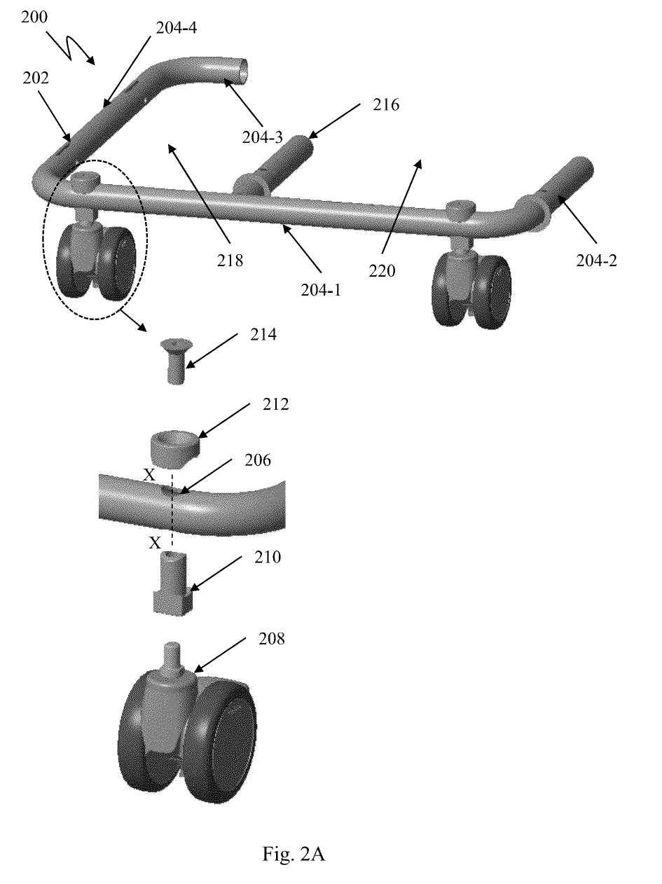

FIGS. 2A and 2B illustrate perspective views of the platform 200 for mounting devices, in accordance with embodiments of the present invention. The platform 200 includes a frame 202 made of one or more arms 204-1, 204-2, 204-3, 204-4 (also referred to as “204” hereinafter) including a plurality of through holes 206 present near edges of the arms 204. FIG. 2A illustrates the frame 202 in its original position. FIG. 2B illustrates the frame 202 in its inverted position. The platform 200 of the present invention allows mounting of the devices on the frame 202, in both the original position and the inverted position, and includes components which are easily reconfigurable to reduce reducing operational inconvenience and time inefficiency in setting up and management of the devices.

The platform 200 includes a plurality of support members 208 configured to support the frame 202 near the edges of the arms 204. Each of the support members 208 includes a hollow shaft 210 having internal threads passing via the through hole 206 of the arm 204.

The support member 208 may be a rolling member such as wheel or a guide roller for supporting movement of the platform 200 mounting the devices. As shown in FIGS. 2A and 2B, the support member 208 may be a caster wheel. The support member 208 may be selected as a bearing pad for providing stability to the platform 200 mounting the devices.

The platform 200 also includes a plurality of spacers 212. Each spacer 212 is disposed on the through hole 206, and includes a bottom surface 212-1 having a profile corresponding to a curvature of corresponding arm 204 of the frame 202. The spacer 212 may be disposed on the arm 204 over a central axis X-X corresponding to the through hole 206.

The platform 200 includes a plurality of threaded plugs 214, each threaded plug 214 passing through the spacer 212 and the through hole 206. The threaded plugs 214 are secured with the internal threads of each of the hollow shaft 210 positioned above the support member 208, to enable coupling of the support member 208 to the frame 202. The frame 202 is invertible. The support member 208, the spacer 212, and the threaded plug 214 are connectable from a top side or a bottom side of the frame 202.

The frame 202 may include a partitioning arm 216 which partitions the frame 202 into a main partition area 218 and an auxiliary partition area 220, such that the devices are to be mounted in the auxiliary partition area 220 of the frame 202. The partitioning arm 216 may prevent mounting of the devices in the main partition area 218.

The platform 200 may also include one or more base plates for mounting of the devices. The base plates may be secured to the frame 202 of the platform 200 such that the devices may be simply placed on the base plates. The base plates may be connected with opposite arms 204 of the frame 202, and may be provided with fitting arrangements to allow fitting of devices on the base plates. The fitting arrangements may include any or a combination of, snap fit arrangements, fixtures, and guided arrangements, such that the devices are snugly fitted on the base plates. According to an embodiment of the present invention, the base plate may be disposed only in the auxiliary partition area 220 of the frame 202 for mounting of the devices.

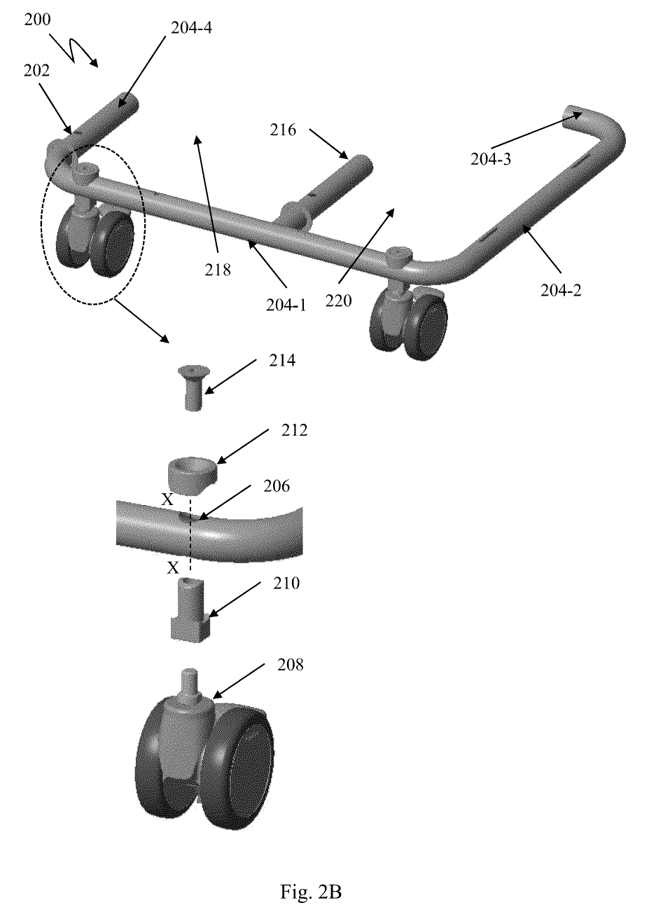

Referring to FIG. 3, where the threaded plug 214 secured with the support member 208 through the spacer 212 is shown. When the threaded plug 214 is secured with the internal threads of hollow shaft 210, a top surface of the threaded plug 214 may be flush with a top surface of the spacer 212, so that so that the top surface of the threaded plug 214 and the top surface of the spacer 212 form a single smooth surface.

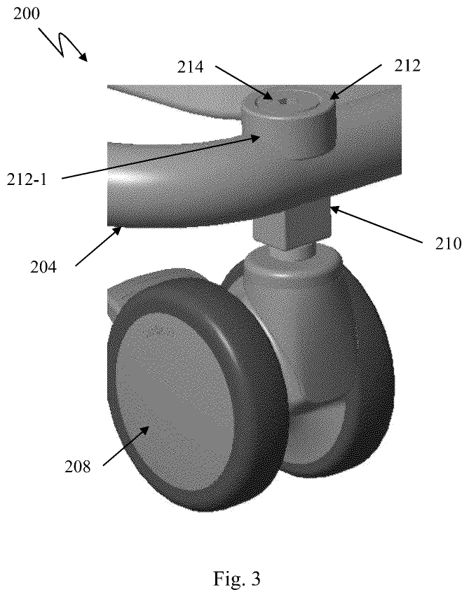

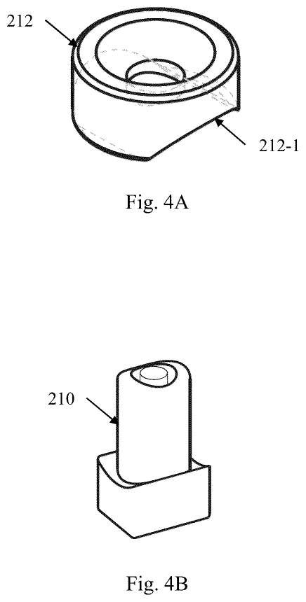

As shown in FIG. 4A, the spacer 212 includes a bottom surface 212-1 having a profile corresponding to curvature of the arm 204. When the arm 204 has a rounded curvature, the bottom surface 212-1 of the spacer 212 may have an arcuate profile corresponding to the rounded curvature of the arm 204. Similarly, when the arm 204 has a rectangular curvature, the bottom surface 212-1 of the spacer 212 may have a rectangular profile corresponding to the rectangular curvature of the arm 204.

FIG. 4B illustrates the shaft 210 of the support member 208 of the platform 200, in accordance with an embodiment of the present invention. As shown, the shaft 210 includes internal threads passing via the through hole 206 of the arm 204, for engaging the threaded plug 214 passing through the spacer 212 and the through hole 206 of the arm 204, in order to enable fitment of the support member 208 with the frame 202 of the platform 200. This allows the support member 208 to efficiently support weight of the platform 200 as well as supporting movement of the platform 200 when the devices are mounted thereon.

The threaded engagement of the threaded plug 214 and the support member 208 allows the threaded plug 214 and the support member 208 to be released or reconfigured when there is a requirement to disassemble and/or reassemble the components of the platform 200. To this effect, when the frame 202 is positioned in an inverted position, i.e., the frame 202 is inverted by 180° in the horizontal plane (see FIG. 2B), the support member 208, the spacer 212 and the threaded plug 214 are reconfigured, to enable the frame 202 to mount the devices in different configurations, i.e., in both original and inverted positions, thereby simplifying the reconfiguring, set up and management of the device.

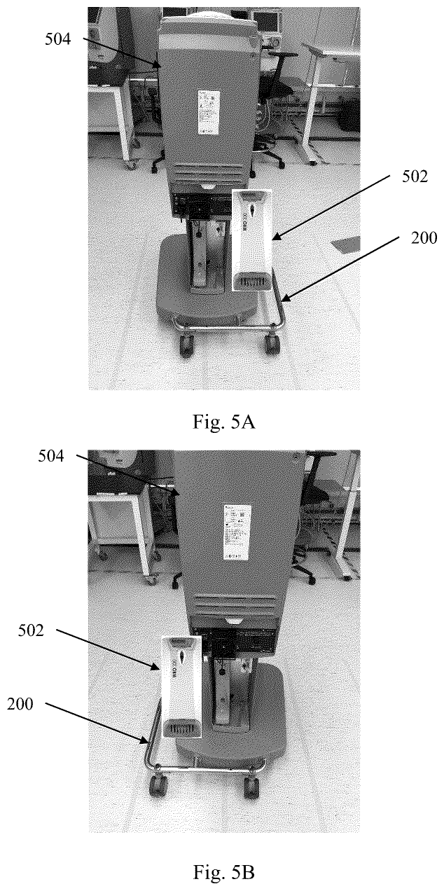

FIGS. 5A and 5B illustrate different configurations of the platform 200 mounting an auxiliary device 502 to be used in conjunction with a primary device 504, in accordance with embodiments of the present invention. The device mounted on the frame 202 of the platform 200 includes the auxiliary device 502 to be used in conjunction with the primary device 504. In an implementation, the primary device 504 may be Baxter® AK 98 Hemodialysis Machine, and the auxiliary device 502 may be Baxter® WRO 300H Reverse Osmosis Unit.

The platform 200 allows mounting of the auxiliary device 502 on the frame 202, in both original position and inverted position of the frame 202, and includes components, such as, support member 208, shaft 210, spacer 212 and threaded plug 214, which are easily reconfigurable to reduce operational inconvenience and time inefficiency in setting up and management of the auxiliary device 502.

As shown in FIG. 5A and SB, when the frame 202 is positioned in the inverted position, the auxiliary device 502 is mounted on the frame 202 of the platform 200 in a first configuration with respect to the primary device 504 which is different from a second configuration when the auxiliary device 502 is mounted on the frame 202 which is positioned in the original position.

In the first configuration, the auxiliary device 502 is mounted on the frame 202 in the original position, such that the auxiliary device 502 is disposed on right side of the primary device 504. On the contrary, in the second configuration, the auxiliary device 502 is mounted on the frame 202 in the inverted position, such that the auxiliary device 502 is disposed on left side of the primary device 504. To this effect, the platform 200 provides for mounting of the auxiliary device 502 in both original as well as inverted positions of the frame 202, thereby simplifying set up and management of the auxiliary device 502 on both sides of the primary device 504 by simply switching between the original position and the inverted position of the frame 202 of the platform 200.

Thus, the present invention provides a platform 200 for mounting one or more devices in different configurations. The components of the platform 200 are reconfigurable such that the frame 202 of the platform 200 is capable of mounting the devices in both original position as well as inverted position, by simply releasing and reconfiguring the components. The platform 200 is used to mount an auxiliary device 502 which is used in conjunction with a primary medical device 504, for instance, in a medical setting, and allows mounting of the auxiliary device 502 on the frame 202, in both original position and inverted position of the frame 202, to reduce operational inconvenience and time inefficiency in setting up and management of the auxiliary device 502. The platform 200 and its components are capable of being retrofitted with conventional medical apparatuses, to improve operational efficiencies and time efficiencies of the conventional medical apparatuses.

In the above detailed description, reference is made to the accompanying drawings that form a part thereof, and illustrate the best mode presently contemplated for carrying out the invention. However, such description should not be considered as any limitation of scope of the present unit. The structure thus conceived in the present description is susceptible of numerous modifications and variations, all the details may furthermore be replaced with elements having technical equivalence.

1.-8. (canceled)

9. A platform for mounting devices comprising:
a frame made of one or more arms comprising a plurality of through holes present near edges of the one or more arms;
a plurality of support members configured to support the frame near the edges of the one or more arms, each support member including a hollow shaft having internal threads passing through at least one of the plurality of through holes;
a plurality of spacers, each spacer being disposed on at least one through hole of the plurality of through holes and comprising a bottom surface having a profile corresponding to a curvature of the frame; and
a plurality of threaded plugs, each threaded plug passing through at least one spacer of the plurality of spacers and the at least one through hole, the threaded plug being secured with the internal threads of the hollow shaft positioned above the support member, to enable coupling of the support member to the frame,
wherein the bottom surface of the at least one spacer has an arcuate profile corresponding to a rounded curvature of the frame,
wherein the support member is a bearing pad for providing stability to the platform mounting the devices,
wherein the devices mounted on the platform include a primary device and at least one auxiliary device to be used in conjunction with the primary device.

10. The platform according to claim 9, further comprising one or more base plates connected with opposite arms of the frame, for mounting the devices.

11. The platform according to claim 9, wherein the plurality of support members are rolling members for supporting movement of the platform mounting the devices.

12. The platform according to claim 9, wherein the frame is invertible, and the plurality of support members, the plurality of spacers, and the plurality of threaded plugs are connectable from a top side or a bottom side of the frame.

13. The platform according to claim 9, wherein a top surface of the threaded plug is flush with a top surface of the at least one spacer.
